(19) United States
(12) Patent Application Publication
(10) Pub. No.: US 2010/0145473 A1
(43) Pub. Date: Jun. 10, 2010

(54) Gradient Template for Angiogensis During Large Organ Regeneration

(76) Inventors: Ioannis V. Yannas, Boston, MA (US); Brendan Harley, Cambridge, MA (US); Christpher J. Zagorski, Essex (GB); Harry K. Reddy, Lexington, MA (US)

Correspondence Address: Pearl Cohen Zedek Latzer, LLP, 1500 Broadway, 12th Floor, New York, NY, 10036, US

(21) Appl. No.: 12/084,569

(22) PCT Filed: Nov. 7, 2006
(86) PCT No.: PCT/US2006/043424

§ 371 Date: Nov. 30, 2009

Related U.S. Application Data
(60) Provisional application No. 60/733,803, filed on Nov. 7, 2005.

(51) Int. Cl. A61F 2/06

(52) U.S. Cl. 623/23.72

(57) ABSTRACT

This invention relates to highly porous scaffolding and methods of producing the same. Specifically, the scaffolding comprises a pore volume fraction of no less than 80% (v/v) of the total volume of the scaffold and interconnecting pores forming channels in the scaffold.

### FIELD OF INVENTION

This invention is directed to highly porous scaffolding and methods of producing the same. Specifically, the scaffolding comprises a pore volume fraction of no less than 80% (v/v) of the total volume of the scaffold and interconnecting pores forming channels in the scaffold.

### BACKGROUND OF THE INVENTION

Implanting a scaffold to regenerate lost or damaged tissue, requires the use of a scaffold that supports adequate cell migration into and around the scaffold, short-term support of these cells following implantation with an adequate supply of oxygen and nutrients and long-term angiogenesis and remodeling of the scaffold (degradation of the scaffold and remodeling of the vasculature and tissue architecture). If all these functions are not supported, new stroma will not be formed and tissue regeneration will not occur.

Scaffolds are prefabricated supports, which may be seeded with cells. While cells can easily adsorb into the outermost portions of the scaffold, cell distributions may not be uniform throughout the scaffold due to random motility and limitations in the diffusion of nutrients. This in turn may lead to uneven and distorted regeneration of tissue, which, if allowed to persist, may create other pathologies. Even if cells are homogenously distributed throughout a large-scale scaffold, there is a need for a vascular supply to nourish the cells in the interior of the scaffold, since these cells are positioned in a location within the scaffold, which is not readily accessible to the surrounding vasculature and are therefore deprived of nutrients and oxygen necessary for their long term viability. Cell survival necessitates it being within the diffusion distance of a capillary, for the formation of a concentration gradient facilitating an exchange whereby the cell can receive an adequate concentration of oxygen and nutrients. While a vascular supply can grow into an implanted scaffold from surrounding vascularized tissue, the angiogenic process takes time, which may result in cell death in the scaffolding interior, prior to adequate vascularization.

One of the limitations to date in successful tissue engineering is a lack of an appropriate material and architecture whereby complex tissues may be assembled, in particular providing the ability of appropriate cells to align themselves in an appropriate configuration to form a functioning tissue.

A major limitation encountered during use of scaffolds in organ replacement is the scale over which formation of new blood vessels (angiogenesis) takes place away from host tissue and inside the scaffold. In one such case, during skin regeneration, synthesis of new tissues takes place primarily inside a full-thickness skin wound, in the plane that characterizes this largely two-dimensional organ (the plane of the epidermis). The scaffold that induces skin synthesis is also a two-dimensional structure that degrades out during completion of synthesis of the new skin. In the process of acquiring most of the structural features and function of normal skin, the newly synthesized organ becomes spontaneously vascularized. In this case, angiogenesis occurs along a path length that does not exceed the thickness of the scaffold, typically with an order of magnitude of about 1 mm.

It is clinically useful to have the ability to regenerate peripheral nerves, synthesize new cylindrical nerve trunk vascularize organs. The new nerve, have formed inside the gap separating two nerve stumps generated following transection of a nerve. Nerve regeneration across a gap of several mm, does not typically occur. The scaffold, which induces nerve regeneration was slender cylinder with diameter about 1 mm and length that typically does not exceed a few mm. In this example as well angiogenesis occurs along a path that does not exceed a few mm, which limits the applicability of the scaffold.

There is a need for a scaffolding capable of supporting tissue regeneration on a large scale, facilitating infiltration of vasculature, allowing access to cells located relatively far below the surface of the scaffold within a period short enough to ensure viability of these cells, as well as support adequate cell migration into and around the scaffold, short-term support of these cells following implantation with an adequate supply of oxygen and nutrients and long-term angiogenesis and remodeling of the scaffold.

### SUMMARY OF THE INVENTION

In one embodiment, the invention provides a solid, porous biodegradable scaffold for implantation in a subject, comprising at least one polymer, and having a pore volume fraction of at least 80% of the total volume of said scaffold, comprising interconnected pores which form channels in said scaffold, wherein
a) said channels have a diameter of between 1-200 μm,
b) a negative gradient in said channel diameter along an axis of said scaffold; and
c) branching of said channels along said axis proportional to said negative gradient

In another embodiment, this invention provides a process for preparing a solid, porous, biocompatible scaffold having branched channels of decreasing diameter, the process comprising the steps of
a) applying a polymeric suspension to a mold comprised of a conductive material, wherein said mold has conical projections disposed at an angle to an axis, said conical projections having diameter between 1-200 μm;
b) super-cooling the suspension-filled mold in (a) in a refrigerant, for a period of time until said suspension is solidified, whereby ice crystals are formed in said solidified polymeric suspension; and
c) removing said mold, thereby exposing said polymeric suspension to sublimation conditions

In another embodiment, this invention provides a scaffold prepared according to a process of the invention.

In one embodiment, this invention provides methods and scaffolding for facilitating or accelerating tissue repair or regeneration, which, in another embodiment, finds application in wound healing.

### DETAILED DESCRIPTION OF THE INVENTION

The invention is directed to solid porous scaffolds, methods of producing the same, and therapeutic applications arising from their utilization.

This invention provides in one embodiment, a scaffold which stimulates or enhances angiogenesis.

Angiogenic scaffolds provide for endothelial cell interaction with a surface of a scaffold which, in turn stimulate formation of blood vessels. In one embodiment. host tissue is replete with blood vessels of many sizes, may serve as the source of endothelial cells for the formation of new vasculature inside the scaffold. In another embodiment, endithelial cells are added with the scaffold, in combination, in yet another embodiment, with compounds desirable for optimal growth in the location and of the tissue type, such as in one embodiment; VEGF. In one embodiment, the invention provides scaffolds, which facilitate blood vessel proliferation and provide spatial guidance for the branching of blood vessels along a desired axis. The combination of proliferative growth and spatial control of such growth allows the organ undergoing synthesis to be perfused with blood through its entire growth sequence. The formation of which represent an embodiment of this invention.

Blood vessel formation involves endothelial cell interaction with extracellular matrix. Extracellular matrices of the present invention that are highly porous, will comprise polymers or graft copolymers, such as (but not limited to) those based on glycoproteins of the extracellular matrix (ECM). In some embodiments, the glycoproteins will comprise collagen, laminin, fibronectin, elastin, proteoglycans, or glycosaminoglycans (GAGs), such as heparin, hyaluronic acid or chondroitin 6-sulfate, or graft coplymers thereof in any combination.

For example, and in one embodiment, the scaffold is comprised of a graft copolymer of a type I collagen and a GAG, whose ratio is controlled by adjusting the mass of the macromolecules mixed to form the copolymer.

In one embodiment, formation of a branching network of blood vessels inside a scaffold of this invention is controlled by the specific surface available for interaction with endothelial cells and with VEGF.

In one embodiment, the invention provides a solid, porous biodegradable scaffold for implantation in a subject, comprising at least one polymer, and having a pore volume fraction of at least 80% of the total volume of said scaffold, comprising interconnected pores which form channels in said scaffold, wherein
a) said channels have a diameter of between 1-200 μm,
b) a negative gradient exists in said channel diameter along an axis of said scaffold; and
c) branching of said channels along said axis proportional to said negative gradient

In one embodiment, the term “scaffold” or “scaffolds” refers to three-dimensional structures that assist in the tissue regeneration process by providing a site for cells to attach, proliferate, differentiate and secrete an extra-cellular matrix, eventually leading to tissue formation. In one embodiment, a scaffold provides a support for the repair, regeneration or generation of a tissue or organ.

In one embodiment, the matrices comprising the scaffold are present in a gradient. In another embodiment, the term “gradient”, when used in reference to a scaffold of the invention refers to a scaffold comprising a material which varies throughout the scaffold, in the concentration of components of which the scaffold is comprised, or in another embodiment, its porosity (which may be reflected in other embodiments in terms of, pore size, pore shape, percent porosity, tortuosity, interconnectivity), or in another embodiment, its cross-link density, or in another embodiment, its density. In one embodiment, pore diameter throughout the scaffold is varied. In another embodiment, any combination of these parameters may be varied in the scaffolds of this invention.

In another embodiment, the scaffold is non-uniformly porous. In one embodiment, the term “porous” refers to a substrate that comprises holes or voids, rendering the material permeable. In one embodiment, non-uniformly porous scaffolds allow for permeability at some regions, and not others, within the scaffold, or in another embodiment, the extent of permeability differs within the scaffold.

In one embodiment, the pores within the scaffold are of a non-uniform average diameter. In another embodiment, the average diameter of said pores varies as a function of its spatial organization in said scaffold, or in another embodiment, average diameter of said pores varies as a function of the pore size distribution along an arbitrary axis of said scaffold.

The scaffolds of the present invention are highly porous, which makes them applicable for tissue engineering, repair or regeneration. Scaffolds with high degree of porosity is useful to facilitate migration of different cell types to the appropriate regions of the scaffold, in one embodiment. In one embodiment, such scaffold facilitates development of appropriate cell-to-cell connections among the cell types comprising the scaffold, required for appropriate structuring of the developing/repairing/regenerating tissue. For example, dendrites or cell processes extension may be accommodated more appropriately via the varied porosity of the scaffolding material.

In another embodiment, the scaffold varies in its average pore diameter and/or distribution thereof. In another embodiment, the average diameter of the pores varies as a function of its spatial organization in said scaffold. In another embodiment, the average diameter of the pores varies as a function of the pore size distribution along an arbitrary axis of the scaffold. In another embodiment, the scaffold comprises regions void of pores. In another embodiment, the regions are impenetrable to molecules greater than 1000 Da in size.

In one embodiment, scaffolds that are non-uniformly porous, vary in their average pore diameter, which may range from 0.75 to 3000 μm, pore size distribution, which may range from about 30 to 200 μM, cross-link density, which may be modified by any crosslinking technology known in the art, or a combination thereof. In one embodiment, average pore diameter may range between about 0.75 to about 3000 μM.

In one embodiment, the scaffold of the invention is biocompatible. In another embodiment, the term “biocompatible” refers to products, which when they break down into elements, such elements are beneficial or in another embodiment, not harmful to the subject or his/its environment. In another embodiment, the term “biocompatible” refers to a material, which tends not to induce fibrosis, inflammatory response, host rejection response, cell adhesion or any combination thereof, following exposure of the scaffold to a subject or cell in said subject. In another embodiment, the term “biocompatible” refers to any substance or compound that has minimal (i.e., no significant difference is seen compared to a control), if any, effect on surrounding cells or tissue exposed to the scaffold in a direct or indirect manner.

In one embodiment, the term “biocompatible” refers to a material which, when placed in a biological tissue, does not provoke a toxic response. In one embodiment, the material which is biocompatible may be organic or in another embodiment synthetic. In one embodiment, the biocompatible material may treat, or in one embodiment, replace, or in another embodiment augment, or in another embodiment stimulate or repair a biological tissue.

In another embodiment, the scaffolds of the invention will comprise a biodegradable polymer. In one embodiment, the term “bioerodible polymer” refers to a water-insoluble polymer that is converted under physiological conditions into water soluble materials without regard to the specific mechanism involved in the erosion process. In one embodiment, “bioerosion” is involved in a physical processes (such as dissolution), or in another embodiment, a chemical processes (such as backbone cleavage). In one embodiment, bioerosion occurs under physiological conditions, yet may be influenced in another embodiment, by high temperature, chemical milieu, etc. in situ. In one embodiment, bioresorption or in another embodiment, bioabsorption indicate that the polymer or in one embodiment, its degradation products are removed by cellular activity (e.g., phagocytosis) in a biological environment. In one embodiment, biocompatible scaffold is bioerodable.

In reference to polymers, the term “degrade” refers in one embodiment to cleavage of the polymer chain, such that the molecular weight stays approximately constant at the oligomer level. In another embodiment, a polymer completely degrade when cleavage of the polymer is at the monomer level such that there is essentially complete mass loss. The term “degrade” refers in one embodiment to “completely degrade”.

In one embodiment, the biodegradable polymers used in the scaffolds and methods of this invention may comprise esters, anhydrides, orthoesters, and amides. Biodegradable polymers, which comprise poly[lactide-co-glycolide], polyanhydrides, and polyorthoesters. In one embodiment, bioerodible polymers comprise polylactides, polyglycolides, and copolymers thereof, poly(ethylene terephthalate), poly(butyric acid), poly(valeric acid), poly(lactide-co-caprolactone), poly(lactide-co-glycolide), polyanhydrides, polyphosphazenes, poly(.epsilon.-caprolactone), poly(dioxanone), poly(hydroxybutyrate), poly(hydroxyvalerate), polyorthoesters, blends, or copolymers thereof. Biodegradable and biocompatible polymers of acrylic and methacrylic acids or esters comprise poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), etc. Other polymers which can be used in the present invention include polyalkylenes such as polyethylene and polypropylene; polyarylalkylenes such as polystyrene; poly(alkylene glycols) such as poly(ethylene glycol); poly(alkylene oxides) such as poly(ethylene oxide); and poly(alkylene terephthalates) such as poly(ethylene terephthalate). Additionally, polyvinyl polymers can be used which include polyvinyl alcohols, polyvinyl ethers, polyvinyl esters, and polyvinyl halides. Exemplary polyvinyl polymers include poly(vinyl acetate), polyvinyl phenol, and polyvinylpyrrolidone. Mixtures of two or more of the above polymers could also be used in the present invention.

In one embodiment the porous, solid scaffold, is a three-dimensional structure with “sponge-like” continuous pores forming an interconnecting network. In another embodiment, the matrix is rigid or in another embodiment, elastic, and it provides a scaffold upon which cells can grow throughout. In one embodiment, its pores are interconnected and provide the continuous network of channels extending through the matrix or in another embodiment permit the flow of nutrients throughout.

In one embodiment the porous, solid scaffold, is a three-dimensional structure with “sponge-like” continuous pores forming an interconnecting network wherein in another embodiment, said scaffold has a surface area of about 20,000 mm²/cm³, with an average pore diameter of about 35 μm and a pore volume fraction of over 90% of the total scaffold volume, or in another embodiment, over 95%, or in another embodiment, over 98% of the total scaffold volume.

In one embodiment, a cell suspension containing cells such as, keratinocytes, chrondocytes and osteoblasts, is injected into the polymer network along with suitable growth factors. The cells are then allowed to grow within the network. As the cells grow the network around them would degrade. The timing of the network degradation coincide in one embodiment with the cells forming their own network (organ/tissue) through inter-cell contacts.

In one embodiment, channel branching in the scaffolds of the invention can be based on the assumption that blood vessels branching increases according to a geometric series (1→2→4→8→16→n, representing a self similarity having a fractal dimension, D=2). Calculations based on this model show that the surface area of the blood vessels as they branch out increases very rapidly as branching proceeds inside the scaffold. In one embodiment, a single blood vessel with radius 100 μm branches out to form, about 64 capillaries with a diameter of about 10 pm, with a concomitant increase in the surface of the treelike vascular network by about 3 folds. A calculation shows that the network formed as in the preceding example requires contact with a scaffold surface of about 20,000 mm²/cm³, corresponding to an average pore diameter of about 35 μm for a scaffold with pore volume fraction over 90%, of the total scaffold volume. In another embodiment, channel branching in the scaffolds of the invention can be based on the assumption that blood vessels branching follow self similarity having a fractal dimension between 1 and 5.

In one embodiment, the term “about” refers to a deviation from the range of 1-20%, or in another embodiment, of 1-10%, or in another embodiment of 1-5%, or in another embodiment, of 5-10%, or in another embodiment, of 10-20%.

In one embodiment, the invention provides a scaffold characterized by a negative gradient in pore size. In one embodiment, branching of the neovasculature inside the scaffold requires a decrease in scaffold pore size (corresponding to an increases in specific surface area) in the direction away from the surface of host tissue, which is a desired effect of the scaffolds of this invention. In another embodiment, the invention provides a scaffold characterized by a pore size of about 200 μm near the host tissue surface and about 20 mm away from the host tissue, the pore size drops to about 100 μm and still further away, at about 40 mm away from the host tissue interface, the pore size drops to 30 μm.

In one embodiment, the invention provide an angiogenic scaffold that induces neovacularization according to an overall architectural pattern that is appropriate for the organ being regenerated. For example, in another embodiment, regeneration of a peripheral nerve along the major nerve axis.

In another embodiment, the formation of a directed network of blood vessels propagating from the surrounding body tissue towards the inside of a scaffold is controlled by the specific surface available for interaction with endothelial cells and with VEGF. In one embodiment, as the blood vessels divide the surface area increases very rapidly, as the pore size decreases. This leads in another embodiment, to a scaffold characterized by an inverse relationship between pore size, and the distance along the axis of maximum desired vascularization. In one embodiment, the pore channels are interconnected to allow for unobstructed vascular, or in another embodiment epithelial propagation.

Therefore, in this aspect of the invention and in another embodiment, the channel diameter is inversely proportional to the distance of the channel from the host tissue.

In one embodiment, the rate of decrease in pore diameter, or in another embodiment channels created by interconnectivity of the pores, is a function of the distance from the periphery of the scaffold, or in another embodiment, from the host tissue surface, or in another embodiment, as a function of the tissue or tissues sought to be regenerated, or in another embodiment, engineered, or in another embodiment treated.

In one embodiment, the invention provides a scaffold comprising a biocompatible material, which, in another embodiment is a carbohydrate, or in another embodiment, single amino acid, or in another embodiment, a monomer of a biocompatible polymer as described herein, or in another embodiment, a combination thereof.

In one embodiment, the invention provides a scaffold comprising at least one polymer, wherein the polymer is a synthetic polymer, or in another embodiment a natural polymer. In another embodiment the scaffold comprises a ceramic, or in another embodiment a metal, or in another embodiment an extracellular matrix protein, or an analogue thereof, or in another embodiment, a combination thereof.

In another embodiment, the polymers of this invention may be copolymers. In another embodiment, the polymers of this invention may be homo- or, in another embodiment heteropolymers. In another embodiment, the polymers of this invention are free radical random copolymers, or, in another embodiment, graft copolymers. In one embodiment, the polymers may comprise proteins, peptides or nucleic acids.

In another embodiment, the polymers may comprise biopolymers such as, for example, collagen. In another embodiment, the polymers may comprise biocompatible polymers such as polyesters of [alpha]-hydroxycarboxylic acids, such as poly(L-lactide) (PLLA) and polyglycolide (PGA); poly-p-dioxanone (PDO); polycaprolactone (PCL); polyvinyl alcohol (PVA); polyethylene oxide (PEO); polymers disclosed in U.S. Pat. Nos. 6,333,029 and 6,355,699; and any other bioresorbable and biocompatible polymer, co-polymer or mixture of polymers or co-polymers described herein.

In another embodiment, the polymers comprising extracellular matrix components may be purified from tissue, by means well known in the art. For example, if collagen is desired, in one embodiment, the naturally occurring extracellular matrix can be treated to remove substantially all materials other than collagen. The purification may be carried out to substantially remove glycoproteins, glycosaminoglycans, proteoglycans, lipids, non-collagenous proteins and nucleic acid (DNA or RNA), by known methods.

In one embodiment, the polymer may comprise Type I collagen, Type II collagen, Type IV collagen, gelatin, agarose, cell-contracted collagen containing proteoglycans, glycosaminoglycans or glycoproteins, fibronectin, laminin, elastin, fibrin, synthetic polymeric fibers made of poly-acids such as polylactic, polyglycolic or polyamino acids, polycaprolactones, polyamino acids, polypeptide gel, copolymers thereof and/or combinations thereof. In one embodiment, the scaffold will be made of such materials so as to be biodegradable.

In another embodiment, the polymers of this invention may be inorganic, and comprise for example, hydroxyapatite, calcium phosphate, alpha-tricalcium phosphate, beta-tricalcium phosphate, calcium carbonate, barium carbonate, calcium sulfate, barium sulfate, polymorphs of calcium phosphate, ceramic particles, or combinations thereof.

In one embodiment, the polymers may comprise a functional group, which enables linkage formation with other molecules of interest, some examples of which are provided further hereinbelow. In one embodiment, the functional group is one, which is suitable for hydrogen bonding (e.g., hydroxyl groups, amino groups, ether linkages, carboxylic acids and esters, and the like).

In another embodiment, functional groups may comprise an organic acid group. In one embodiment, the term “organic acid group” is meant to include any groupings which contain an organic acidic ionizable hydrogen, such as carboxylic and sulfonic acid groups. The expression “organic acid functional groups” is meant to include any groups which function in a similar manner to organic acid groups under the reaction conditions, for instance metal salts of such acid groups, such as, for example alkali metal salts like lithium, sodium and potassium salts, or alkaline earth metal salts like calcium or magnesium salts, or quaternary amine salts of such acid groups, such as, for example quaternary ammonium salts.

In one embodiment, functional groups may comprise acid-hydrolyzable bonds including ortho-ester or amide groups. In another embodiment, functional groups may comprise base-hydrolyzable bonds including alpha-ester or anhydride groups. In another embodiment, functional groups may comprise both acid- or base-hydrolyzable bonds including carbonate, ester, or iminocarbonate groups. In another embodiment, functional groups may comprise labile bonds, which are known in the art and can be readily employed in the methods/processes and scaffolds described herein (see, e.g. Peterson et al., Biochem. Biophys. Res. Comm. 200 (3): 1586-159 (1994) 1 and Fred et al., J. Med. Chem. 43: 4319-4327 (2000)).

In another embodiment, the scaffold further comprises a pH-modifying compound. In one embodiment, the term “pH-modifying” refers to an ability of the compound to change the pH of an aqueous environment when the compound is placed in or dissolved in that environment. The pH-modifying compound, in another embodiment, is capable of accelerating the hydrolysis of the hydrolyzable bonds in the polymer upon exposure of the polymer to moisture and/or heat. In one embodiment, the pH-modifying compound is substantially water-insoluble. Suitable substantially water-insoluble pH-modifying compounds may include substantially water-insoluble acids and bases. Inorganic and organic acids or bases may be used, in other embodiments.

In one embodiment, the scaffolds of this invention comprise a collagen, a glycosaminoglycan, or a combination thereof.

In one embodiment of the invention, scaffolds may vary in terms of their cross-link density. In one embodiment the term “cross link density” refers to the average number of monomers between each cross-link. In another embodiment, the lower the number of monomers between cross links, the higher the cross link density, which, in another embodiment affects the physic-chemical properties of the scaffold. The cross-linking density should be controlled in one embodiment, so as to obtain a pore size large enough to allow cell migration, or in another embodiment any combination of properties desired.

In another embodiment, pore size may be determined by scanning electron microscopy or in another embodiment, by using macromolecular probes.

In one embodiment, the invention provides a scaffold, wherein the cross-link density of the scaffold is modified by exposing the scaffold to a cross-linking agent, or in another embodiment, to a cross-linking process. In one embodiment, the cross-linking agent is glutaraldehyde, or in another embodiment formaldehyde, or in another embodiment paraformaldehyde, or in another embodiment formalin, (1 ethyl 3-(3dimethyl aminopropyl)carbodiimide (EDAC), or in another embodiment UV light, or in another embodiment, a combination thereof. In one embodiment the exposure time vary to control the cross-link density as described hereinabove. In one embodiment, super-cooling the polymeric suspension under conditions inducing a gradient as described hereinbelow, creates a scaffold wherein the cross link density varies throughout the scaffold.

In one embodiment, as described herein, other molecules may be incorporated within the scaffold, which may, in another embodiment, be attached via a functional group, as herein described. In another embodiment, the molecule is conjugated directly to the scaffold.

In another embodiment, the scaffolds may comprise ECM components, such as hyaluronic acid and/or its salts, such as sodium hyaluronate; dermatan sulfate, heparan sulfate, chondroiton sulfate and/or keratan sulfate; mucinous glycoproteins (e.g., lubricin), vitronectin, tribonectins, surface-active phospholipids, rooster comb or umbilical hyaluronate. In some embodiments, the extracellular matrix components may be obtained from commercial sources, such as ARTHREASETM high molecular weight sodium hyaluronate; SYNVISC® Hylan G-F 20; HYLAGAN® sodium hyaluronate; HEALON® sodium hyaluronate and SIGMA® chondroitin 6-sulfate.

In another embodiment, one or more biomolecules may be incorporated in the scaffold. The biomolecules may comprise, in other embodiments, drugs, hormones, antibiotics, antimicrobial substances, dyes, radioactive substances, fluorescent substances, silicone elastomers, acetal, polyurethanes, radiopaque filaments or substances, anti-bacterial substances, chemicals or agents, including any combinations thereof. The substances may be used to enhance treatment effects, reduce the potential for implantable article erosion or rejection by the body, enhance visualization, indicate proper orientation, resist infection, promote healing, increase softness or any other desirable effect.

In one embodiment, the scaffold varies in terms of its polymer concentration, or concentration of and component of the scaffold, including biomolecules and/or cells incorporated within the scaffold.

In another embodiment, the biomolecules may comprise angiogenic factors Angiogenic factors include platelet derived growth factor (PDGF), vascular endothelial growth factor (VEGF), basic fibroblast growth factor (bFGF), bFGF-2, leptins, plasminogen activators (tPA, uPA), angiopoietins, lipoprotein A, transforming growth factor-.beta., bradykinin, angiogenic oligosaccharides (e.g., hyaluronan, heparan sulphate), thrombospondin, hepatocyte growth factor (also known as scatter factor) and members of the CXC chemokine receptor family. Anti-inflammatory factors comprise steroidal and non-steroidal compounds and examples include: Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetiydamine; Tiopinac; Tixocortol Pivalate; Tolmetin; Tolmetin Sodium; Triclonide; Triflumidate; Zidometacin; Zomepirac Sodium

In one embodiment, the biomolecule may comprise chemotactic agents; antibiotics, steroidal or non-steroidal analgesics, anti-inflammatories, immunosuppressants, anti-cancer drugs, various proteins (e.g., short chain peptides, bone morphogenic proteins, glycoprotein and lipoprotein); cell attachment mediators; biologically active ligands; integrin binding sequence; ligands; various growth and/or differentiation agents (e.g., epidermal growth factor, IGF-I, IGF-II, TGF-β I-III, growth and differentiation factors, vascular endothelial growth factors, fibroblast growth factors, platelet derived growth factors, insulin derived growth factor and transforming growth factors, parathyroid hormone, parathyroid hormone related peptide, bFGF; TGFβ superfamily factors; BMP-2; BMP-4; BMP-6; BMP-12; sonic hedgehog; GDF5; GDF6; GDF8; PDGF); small molecules that affect the upregulation of specific growth factors; tenascin-C; hyaluronic acid; chondroitin sulfate; fibronectin; decorin; thromboelastin; thrombin-derived peptides; heparin-binding domains; heparin; heparan sulfate; DNA fragments, DNA plasmids, or any combination thereof.

In one embodiment, the scaffold varies in terms of its polymer concentration, or concentration of and component of the scaffold, including biomolecules and/or cells incorporated within the scaffold.

In another embodiment, the scaffold may comprise one or more of an autograft, an allograft and a xenograft of any tissue with respect to the subject.

In one embodiment, the scaffolds may comprise cells. In one embodiment, the cells may include one or more of the following: chondrocytes; fibrochondrocytes; osteocytes; osteoblasts; osteoclasts; synoviocytes; bone marrow cells; mesenchymal cells; stromal cells; stem cells; embryonic stem cells; precursor cells derived from adipose tissue; peripheral blood progenitor cells; stem cells isolated from adult tissue; genetically transformed cells; a combination of chondrocytes and other cells; a combination of osteocytes and other cells; a combination of synoviocytes and other cells; a combination of bone marrow cells and other cells; a combination of mesenchymal cells and other cells; a combination of stromal cells and other cells; a combination of stem cells and other cells; a combination of embryonic stem cells and other cells; a combination of precursor cells isolated from adult tissue and other cells; a combination of peripheral blood progenitor cells and other cells; a combination of stem cells isolated from adult tissue and other cells; and a combination of genetically transformed cells and other cells.

In one embodiment, cells may be engineered to express growth factors which, in another embodiment promote optimal tissue engineering.

In one embodiment, the cells incorporated into the scaffold and methods of the invention are keratinocytes, or chrondocytes and osteoblasts in other embodiments. In another embodiment, the incorporated cells express the same or different growth factors, which may be specific for the tissue sought to be regenerated, repaired or engineered.

In one embodiment, the invention provides a scaffold, wherein said scaffold is oriented such that regions of said scaffold with a larger pore diameter are placed proximally and regions with a smallest pore diameter are placed more distally to a site of said implantation in said subject.

In one embodiment, the size and shape of said scaffold is a function of the tissue into which the scaffold is to be implanted. In another embodiment, the scaffold, when implanted, promotes angiogenesis within, or proximal to the scaffold.

In one embodiment the scaffold is comprised of a material whose stiffness is sufficient to resist compressive forces of tissue proximal to a site of implantation. In another embodiment, degree of cross-linking of the scaffold material is adjusted to compensate for the compressive forces of the surrounding tissue. In one embodiment, plasticizers are embedded in the scaffold to allow imparting some elasticity to the scaffold, without collapse of the folds in the surface of the scaffold, which, in another embodiment will vary in depth and width depending on the compressive force of the surrounding target tissue.

In one embodiment, the scaffold is fabricated using a process that creates an amorphous glassy-state solid, comprised of a biocompatible polymer. In one embodiment “glassy-state solid” refers to an amorphous metastable solid wherein rapid removal of a plasticizer causes increase in viscosity of the biopolymer to the point where translational mobility of the critical polymer segment length is arrested and allignment corresponding to the polymer's inherent adiabatic expansion coefficient is discontinued.

In another embodiment, the plasticizer may be any substance of molecular weight lower than that of the biocompatible polymer that creates an increase in the free interstitial volume. In one embodiment, the plasticizer is an organic compound, which in one embodiment is triglyceride of varying chain length, or in another embodiment, the plasticizer is water.

In one embodiment, amorphous glassy-state solid is accomplished by rapid cooling of an aerated melt of the biocompatible polymer, or in another embodiment by rapid solvent removal under vacuum, or in another embodiment, by freeze-drying. In one embodiment, amorphous glassy-state solid is accomplished by extrusion, which in one embodiment is at temperatures higher than 65° C. or, in another embodiment, at temperatures between about 4 and about 40° C. In one embodiment, width, length, depth, or a combination thereof, of the surface folds are designed into the dye used for extrusion, in conjunction with extrusion conditions. It will be understood by a skilled person in the art, that any process capable of producing amorphous glass with high portion of interconnected porosity (sponge-like) where pore size is controllable by varying the fabrication conditions is appropriate for use for producing a scaffold of this invention and is thus within the scope of the invention.

In one embodiment, scaffolds are prepared according to the processes of this invention, in a highly porous form, by freeze-drying and sublimating the material. This can be accomplished by any number of means well known to one skilled in the art, such as, for example, that disclosed in U.S. Pat. No. 4,522,753 to Dagalakis, et al. For examples, porous gradient scaffolds may be accomplished by lyophilization. In one embodiment, extracellular matrix material may be suspended in a liquid. The suspension is then frozen and subsequently lyophilized. Freezing the suspension causes the formation of ice crystals from the liquid. These ice crystals are then sublimed under vacuum during the lyophilization process thereby leaving interstices in the material in the spaces previously occupied by the ice crystals. The material density and pore size of the resultant scaffold may be varied by controlling, in other embodiments, the rate of freezing of the suspension and/or the amount of water in which the extracellular matrix material is suspended at the initiation of the freezing process.

According to this aspect of the invention and in one embodiment, to produce scaffolds having a relatively large, uniform pore size and a relatively low material density, the extracellular matrix suspension may be frozen at a slow, controlled rate (e.g., −1° C./min or less) to a temperature of about −20° C., followed by lyophilization of the resultant mass. To produce scaffolds having a relatively small uniform pore size and a relatively high material density, the extracellular matrix material may be tightly compacted by centrifuging the material to remove a portion of the liquid (e.g., water) in a substantially uniform manner prior to freezing. Thereafter, the resultant mass of extracellular matrix material is flash-frozen using liquid nitrogen followed by lyophilization of the mass. To produce scaffolds having a moderate uniform pore size and a moderate material density, the extracellular matrix material is frozen at a relatively fast rate (e.g., >−1° C./min) to a temperature in the range of −20 to −40° C. followed by lyophilization of the mass.

In another embodiment, this invention provides a process for preparing a solid, porous, biocompatible scaffold having branched channels of decreasing diameter, the process comprising the steps of applying a polymeric suspension to a mold comprised of a conductive material, wherein said mold has conical projections disposed at an angle to an axis, said conical projections having diameter between 1-200 μm; super-cooling the suspension-filled mold in a refrigerant, for a period of time until said suspension is solidified, whereby ice crystals are formed in said solidified polymeric suspension; and removing the conical projections from said solidified polimeric suspension, thereby exposing said polimeric suspension to sublimation conditions.

In one embodiment, “polymeric suspnsion” or “suspension” refers to any suspended system that would form a solid scaffold upon removal of one phase in the system. In one embodiment, the suspended system is a suspension, or in another embodiment emulsion, or in another embodiment, gel or in another embodiment, foam, or in another embodiment a thermodynamically incompatible polymer mixture. In one embodiment, the polymeric suspension is comprised of monomers or in another embodiment, single biocompatible molecules.

According to this aspect of the invention, and in another embodiment, in order to produce scaffolding of this invention, solar bath effect is used to control ice crystallization rate and size thereby controlling pore size in the lyophilized mass. In one embodiment, a solute is incorporated into the mass and a temperature gradient is induced by placing the pan containing the mass on a cold plate, which in one embodiment may be the freeze-dryer shelf, or in another embodiment a heat lamp may be placed on top of the pan. Since solubility is a function of temperature, a solute concentration gradient will result. In another embodiment, solute concentration affects the freezing temperature, resulting in different crystal size in a fixed freezing time, which, in a gradually concentrated solute will result in graduated porosity with pore size inversely proportional to the direction of increased solute concentration. In one embodiment, the solute comprises heterogeneous nucleation centers for water.

In another embodiment of the invention, the term “projections” or “projection” refers to mold extensions, extending from the mold into the mold volume, where the polymeric suspension can not solidify. In one embodiment, these extensions are oriented along an axis which is desired for the effective use of the scaffolds of the invention. In one embodiment, the conical extensions have a diameter of no more than 200 μm at the wide base and no less than 1 μm at the narrow end of the conical extensions. In one embodiment, the extensions are branched to emulate the desired final branching of the blood vessels sought to be generated. In another embodiment, the extensions are made from a biodegradable material and can be degraded without affecting the biodegradable material of the scaffold surrounding the extensions, such as in one embodiment, the extensions are made of paraffin and following sublimation of the water in the scaffold, the extensions are removed by emersion of the scaffold in an organic solvent (such as hexane). In one embodiment, the projections are made of a conductive material and are removed prior to sublimation.

In one embodiment, the gradient is preserved by halting the freezing process prior to achieving thermodynamic equilibrium. The means for determining the time to achieving thermodynamic equilibrium in a slurry thus immersed, when in a container with a given geometry, will be readily understood by one skilled in the art. Upon achieving the desired temperature gradient, the slurry, in one embodiment, is removed from the bath and subjected to freeze-drying. Upon sublimation, the remaining material is the scaffolding comprising the polymer, with a gradient in its average pore diameter.

In another embodiment, a gradient in freezing rate of the scaffold is generated with the use of a graded thermal insulation layer between the container, which contains the scaffold components, and a shelf in a freezer on which the container is placed. In one embodiment, a gradient in the thermal insulation layer is constructed via any number of means, well known in the art, such as, for example, the construction of a thicker region in the layer along a particular direction, or in another embodiment, by varying thermal conductivity in the layer. The latter may be accomplished via use of, for example, aluminum and copper, or plexiglass and aluminum, and others, all of which represent embodiments of the present invention.

In one embodiment, the invention provides a scaffold prepared according to the process described herein.

According to this aspect of the invention, and in one embodiment, the process further comprises the step of exposing the scaffold to a temperature gradient, or in another embodiment to solutions with cross-linking agent gradient.

In one embodiment, the invention provides a method of organ or tissue engineering in a subject, comprising the step of implanting a scaffold of any of the embodiments mentioned herein.

In another embodiment, this invention provides a method of organ or tissue repair or regeneration in a subject, comprising the step of implanting a scaffold of this invention in a subject.

According to these aspects of the invention, and in one embodiment, the scaffold may be one produced by a process of this invention.

In one embodiment, use of the scaffolds for repair, regeneration of tissue is in cases where native tissue is damaged, in one embodiment, by trauma, or in another embodiment, compounded by diabetes. In another embodiment, the gradient scaffold allows for incorporation of individual cells, which are desired to be present in the developing/repairing/regenerating tissue.

According to these aspects of the invention, and in one embodiment, the method further comprises the step of implanting cells in the subject. In one embodiment, the cells are seeded on said scaffold, or in another embodiment, on the periphery of the scaffold. In another embodiment, the cells are stem or progenitor cells. In another embodiment, the method further comprises the step of administering cytokines, growth factors, hormones or a combination thereof to the subject. In another embodiment, the engineered organ or tissue is comprised of heterogeneous cell types. In another embodiment, the engineered organ or tissue is a connector organ or tissue, which in another embodiment, is a tendon or ligament. In one embodiment, the tissue is breast tissue, or in another embodiment skin tissue.

As can be seen from the forgoing description, the concepts of the present disclosure provide numerous advantages. For example, the concepts of the present disclosure provide for the fabrication of an implantable gradient scaffold, which may have varying mechanical properties to fit the needs of a given scaffold design. For instance, the pore size and the material density may be varied to produce a scaffold having a desired mechanical configuration. In particular, such variation of the pore size and the material density of the scaffold is particularly useful when designing a scaffold which provides for a desired amount of cellular migration therethrough, while also providing a desired amount of structural rigidity. In addition, according to the concepts of the present disclosure, implantable devices can be produced that not only have the appropriate physical microstructure to enable desired cellular activity upon implantation, but also has the biochemistry (collagens, growth factors, glycosaminoglycans, etc.) naturally found in tissues where the scaffolding is implanted for applications such as, for example, tissue repair, tissue regneration, angiogenesis or nerve regeneration.

In one embodiment, the scaffold is cultured for a period of time prior to implantation in the subject. In another embodiment, cells are seeded at the periphery of said scaffold, wherein, in one embodiment, cells are stem or progenitor cells.

In another embodiment, the porous solid scaffold having seeded progenitor cells, with or without their progeny, is impregnated with a gelatinous agent that occupies pores of the matrix. In one embodiment, the term “seeded” refers to the fact that progenitor cells, with or without their progeny, are seeded prior to, substantially at the same time as, or following impregnation (or infiltration) with a gelatinous agent. For example, in another embodiment, the cells may be mixed with the gelatinous agent and seeded at the same time as the the impregnation of the matrix with the agent. In another embodiment, the progenitor or stem cells, with or without their progeny, are pre-seeded onto the porous solid matrix. According to the invention, and in one embodiment an amount of the cells is introduced in vitro into the porous solid scaffold, and cultured in an environment that is free of inoculated stromal cells, stromal cell conditioned medium, and exogenously added hematopoietic growth factors that promote hematopoietic cell maintenance, expansion and/or differentiation, other than serum.

In one embodiment, the scaffold is implanted proximally to a host tissue surface, with an orientation such that regions of said scaffold with a larger pore diameter are placed proximally to the host's tissue surface and regions with a smallest pore diameter are placed more distally to said host tissue surface. In another embodiment, pore diameter is inversely proportional to the distance of the pore, or in one embodiment channel, from the host tissue surface. In one embodiment at about 20 mm away from the host tissue surface, said pore diameter is about 100 μm, or in another embodiment, at about 40 mm away from said host tissue surface, said pore diameter is about 30 μm.

The following examples are presented in order to more fully illustrate the preferred embodiments of the invention. They should in no way be construed, however, as limiting the broad scope of the invention.

### EXAMPLES

### Example 1

### Regeneration of A Large 3D Volume of Breast Tissue

The scaffold used is a sphere which is 50 mm in diameter, the pore structure form open channels at or near the surface which extend to the center of the scaffold. The diameter of these channels increases from the center to the scaffold surface, with the diameter near the surface as high as a few millimeters. As the channels extend toward the center they may divide to form a network of channels inside the scaffold, mimicking the progressive division of blood vessels in tissue. The scaffold is seeded with appropriate cells in the periphery. The cells extend from the outer surface to an approximate depth of 10 mm inside the scaffold. The scaffold also has VEGF bound onto the collagen fibers. The diameter of the pore channels at the scaffold surface is 1 mm.

The procedure for implanting the device is analogous to the procedure used to implant saline-filled breast implants. The scaffold is inserted by using a trans-axillary approach. The device is placed above the pectoralis major muscle, as placement below would expose the scaffold to a different cellular environment to the tissue types being regenerated. Surgeons also believe that placement below pectoralis major reduces capsular contracture, utilized to help bring the vascular bed in close proximity with the scaffold surface. The patient is placed under general anesthetic. Following the axillary incision the surgeon creates a small pocket to insert the scaffold between the breast gland tissue and pectoralis major. The scaffold is inserted into the space formed and the incision is closed. Following surgery the patient wears a specially designed undergarment to protect the device from being dislodged and from excessive compressive force. Pain medications are utilized as necessary following surgery. Once in place, the pressure from the surrounding tissue brings the existing vasculature in contact with the device's outer surface, forcing tissue into the invagination. The formation of capsule around the implant occurs spontaneously, creating multiple layers of fibrous tissue containing a variable amount of contractile cells, the innermost layers contain vasculature which is brought in close proximity to the scaffold. The degree to which capsule forms around the implant is dependant on the material from which it is composed, forming more around synthetic polymers.

Inflammatory exudate is released from capillaries in phases, bathing the wound in plasma proteins. Different cell types are recruited over different phases of time to remove damaged tissue, induce the formation of new tissue, reconstruct damaged matrix, basement membrane and connective tissue, and establish a new blood supply. Fluid exudate is released in three phases following injury: the first phase begins almost immediately after injury and involves a histamine-stimulated release of fluid and lasts anywhere between 8 to 30 minutes. The next phase is similar beginning straight after the first; it lasts longer, up to several days. The final phase commences a few hours after injury and the effects become maximal in 2-3 days, gradually resolving over a matter of weeks. Cellular exudate is produced in the second and third phases. The general make-up of the matrix becomes more fluid, allowing the contents of the exudate to diffuse more easily, but a sudden increase in tissue pressure doesn't occur. This will help exudate flow through the pores and channels in the scaffold, without a sudden increase in pressure damaging the implant. The components of exudate, both cellular and molecular (as detailed earlier) aid in angiogenesis and the regeneration of tissue.

The inflammatory exudate starts to flow into the large open pore channels at the scaffold surface within the first few hours following implantation. Fibrin, formed from fibrinogen condenses, creating a network on which blood vessels can grow. Other factors and cells present in exudate help reconstruct the stroma within the scaffold and promote angiogenesis. The vasculature in the pre-existing tissue comes closer to the scaffold due to contraction of the surrounding tissue and increased pressure from the space taken up by the implant. An additional vascular network is formed surrounding the scaffold as capsule forms. The high concentration of angiogenic factors in exudate and from migrating/seeded cells causes blood vessels to grow into the scaffold, supporting the nearby cells indefinitely.

The invaginations in the scaffolds structure decrease the distance blood vessels travel to reach the center of the scaffold. They also decrease the amount of blood vessel growth required to vascularize the outer regions of the scaffold, thus rapidly vascularizing a large proportion of the volume of the implant (since x mm of blood vessel growth toward the surface fills a greater volume of scaffold than it would nearer the center).

The phase of cell proliferation begins early on at around 24-48 hours, peaking at around 2-3 weeks. Tissue remodeling begins from around 1-2 weeks. Near complete degradation of the scaffold and tissue regeneration is achieved within 4 weeks.

### Example 2

### Freeze-Sublimation Methods For Constructing Gradient Scaffolding With Varied Pore Diameter

### Preparation of Slurry

Extracellular matrix components, such as, for example, microfibriallar, type I collagen, isolated from bovine tendon (Integra LifeSciences) and chondroitin 6-sulfate, isolated from shark cartilage (Sigma-Aldrich), 10% (w/w) at 1:1 ratio are combined with 0.05M acetic acid at a pH ˜3.2 are mixed at 15, 000 rpm, at 4° C., then degassed under vacuum at 50 mTorr.

### Varying Pore Diameter

The suspension is placed in a container, and only part of the container (up to 10% of the length) is submerged in a supercooled silicone bath. The equilibration time for freezing of the slurry is determined, and the freezing process is stopped prior to achieving thermal equilibrium. The container is then removed from the bath and the slurry is then sublimated via freeze-drying (for example, VirTis Genesis freeze-dryer, Gardiner, NY). Thus, a thermal gradient occurs in the slurry, creating a freezing front, which is stopped prior to thermal equilibrium, at which point freeze-drying is conducted, causing sublimation, resulting in a matrix copolymer with a graded average pore diameter field.

In another method, the suspension is placed in a container, on a freezer shelf, where a graded thermal insulation layer is placed between the container and the shelf, which also results in the production of a gradient freezing front, as described above. The graded thermal insulation layer can be constructed by any number of means, including use of materials with varying thermal conductivity, such as aluminum and copper, or aluminum and plexiglass, and others.

In one embodiment, the container is a multicomponent mold, containing removable elements. In one embodiment, removal of these elements creates tunnels within the frozen slurry. In another embodiment, the removable elements have conical shape, such that in one embodiment, the tunnel diameter narrows the further the distance is from the periphery of the scaffold.

In one embodiment, the surface of the mold creates indentations and channels in the frozen slurry, thereby creating surface folds of desired geometry and distribution across

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A solid, porous biodegradable scaffold for implantation in a subject, comprising at least one polymer, and having a pore volume fraction of at least 80% of the total volume of said scaffold, comprising interconnected pores which form channels in said scaffold, wherein
a. said channels have a diameter of between 1-200 μm,
b. a negative gradient exists in said channel diameter along an axis of said scaffold; and
c. branching of said channels along said axis is proportional to said negative gradient

2. The scaffold of claim 1, wherein said channel diameter is inversely proportional to the distance of said channel from the host tissue.

3. The scaffold of claim 1, wherein said polymer comprises at least one synthetic or natural polymer, ceramic, metal, extracellular matrix protein or an analogue thereof.

4. The scaffold of claim 3, wherein said extracellular matrix proteins comprise a collagen, a glycosaminoglycan, or a combination thereof.

5. The scaffold of claim 1, wherein said scaffold varies in its cross-link density, which may be modified by any crosslinking technology known in the art.

6. The scaffold of claim 5, wherein said cross-linking agent is glutaraldehyde, formaldehyde, paraformaldehyde, formalin, (1 ethyl 3-(3dimethyl aminopropyl)carbodiimide (EDAC), or UV light, or a combination thereof.

7. The scaffold of claim 1, wherein said scaffold further comprises cells, extracellular matrix components, growth factors, cytokines, hormones, inflammatory stimuli, angiogenic factors, or a combination thereof.

8. The scaffold of claim 1, wherein the size and shape of said scaffold is a function of the tissue into which the scaffold is to be implanted.

9. The scaffold of claim 1, wherein said scaffold, when implanted, promotes angiogenesis within, or proximal to the scaffold.

10. The scaffold of claim 1, wherein said scaffold is comprised of a material whose stiffness is sufficient to resist compressive forces of tissue proximal to a site of implantation.

11. The scaffold of claim 1, wherein said pores have a diameter ranging from 30 μm-200 μm.

12. The scaffold of claim 11, wherein said scaffold is oriented such that regions of said scaffold with a larger pore diameter are placed proximally and regions with a smallest pore diameter are placed more distally to a site of said implantation in said subject.

13. The scaffold of claim 1, wherein said scaffold has a surface area of about 20,000 mm²/cm³, with an average pore diameter of about 35 μm and a pore volume fraction of over 90%.

14. A process for preparing a solid, porous, biodegradable scaffold having branched channels of decreasing diameter, the process comprising the steps of
a) applying a polymeric suspension to a mold comprised of a conductive material, wherein said mold has conical projections disposed at an angle to an axis, said conical projections having diameter between 1-200 μm;
b) super-cooling the suspension-filled mold in (a) in a refrigerant, for a period of time until said suspension is solidified, whereby ice crystals are formed in said solidified polymeric suspension; and
c) removing the conical projections from said solidified polimeric suspension, thereby exposing said polimeric suspension to sublimation conditions.

15. A scaffold, prepared according to the process of claim 14.

16. The scaffold of claim 15, wherein said pore volume is no less than 80%

17. A method of organ or tissue engineering in a subject, comprising the step of implanting a scaffold of claim 1 in said subject.

18. The method of claim 17, further comprising the step of implanting cells in said subject.

19. The method of claim 18, wherein said cells are seeded on said scaffold.

20. The method of claim 19, wherein said scaffold is cultured for a period of time prior to implantation in said subject.

21. The method of claim 19, wherein said cells are seeded at the periphery of said scaffold.

22. The method of claim 19, wherein said cells are stem or progenitor cells.

23. The method of claim 19, wherein said cells are engineered to express extracellular matrix components, growth factors, cytokines, hormones, inflammatory stimuli, angiogenic factors, or a combination thereof.

24. The method of claim 17, wherein said engineering is of an organ or tissue comprised of heterogeneous cell types.

25. The method of claim 17, wherein angiogenesis is stimulated within said scaffold.

26. The method of claim 17, wherein said scaffold comprises pores having a diameter ranging from 30 μm-200 μm.

27. The method of claim 25, wherein said scaffold is implanted proximally to a host tissue surface, with an orientation such that regions of said scaffold with a larger pore diameter are placed proximally and regions with a smallest pore diameter are placed more distally to said host tissue surface.

28. The method of claim 25, wherein at about 20 mm away from said host tissue surface, said pore diameter is about 100 μm.

29. The method of claim 25, wherein at about 40 mm away from said host tissue surface, said pore diameter is about 30 μm.

30. The method of claim 17, wherein said scaffold has a surface area of 20,000 mm²/cm³, with an average pore diameter of about 35 μm and a pore volume fraction of over 90%.

31. A method of organ or tissue repair or regeneration in a subject, comprising the step of implanting a scaffold of claim 1 in said subject.

32. The method of claim 31, further comprising the step of implanting cells in said subject.

33. The method of claim 32, wherein said cells are seeded on said scaffold.

34. The method of claim 32, wherein said scaffold is cultured for a period of time prior to implantation in said subject.

35. The method of claim 32, wherein said cells are seeded at the periphery of said scaffold.

36. The method of claim 32, wherein said cells are stem or progenitor cells.

37. The method of claim 32, wherein said cells are engineered to express extracellular matrix components, growth factors, cytokines, hormones, inflammatory stimuli, angiogenic factors, or a combination thereof.

38. The method of claim 31, wherein said engineering is of an organ or tissue comprised of heterogeneous cell types.

39. The method of claim 31, wherein angiogenesis is stimulated within said scaffold.

40. The method of claim 31, wherein said scaffold comprises pores having a diameter ranging from 30 μm-200 μm.

41. The method of claim 40, wherein said scaffold is implanted proximally to a host tissue surface, with an orientation such that regions of said scaffold with a larger pore diameter are placed proximally and regions with a smallest pore diameter are placed more distally to said host tissue surface.

42. The method of claim 41, wherein at about 20 mm away from said host tissue surface, said pore diameter is about 100 μm.

43. The method of claim 41, wherein at about 40 mm away from said host tissue surface, said pore diameter is about 30 μm.

44. The method of claim 31, wherein said scaffold has a surface area of 20,000 mm²/cm³, with an average pore diameter of about 35 μm and a pore volume fraction of over 90%.
